(19) United States
(12) Patent Application Publication
(10) Pub. No.: US 2023/0355774 A1
(43) Pub. Date: Nov. 9, 2023

(54) COMPOSITIONS CONTAINING COATED MINERALS OR MINERAL-AMINO ACID COMPLEXES

(71) Applicant: Melaleuca, Inc., Idaho Falls, ID (US)

(72) Inventors: Nasser A. Fredj, Ammon, ID (US); Ricardo Gutierrez, Idaho Falls, ID (US)

(21) Appl. No.: 18/143,799

(22) Filed: May 5, 2023

Related U.S. Application Data
(60) Provisional application No. 63/339,044, filed on May 6, 2022.

(51) Int. Cl. A61K 47/54; A61K 47/69; A61K 45/06

(52) U.S. Cl. CPC A61K 47/549; A61K 47/6941; A61K 45/06

(57) ABSTRACT

This document provides compositions containing mineral-amino acid/polysaccharide complexes, where at least one of the complexes in the composition has a slow release coating, and in some cases, other complexes in the composition do not have a slow release coating. This document also provides and methods of making and using the complexes and the compositions.

### CROSS-REFERENCE TO RELATED APPLICATIONS

This application claims priority from U.S. Provisional Application Ser. No. 63/339,044, filed May 6, 2022. The disclosure of the prior application is considered part of (and is incorporated by reference in) the disclosure of this application.

### TECHNICAL FIELD

This document relates to compositions containing minerals or complexes containing a mineral-amino acid compound, optionally coupled to a polysaccharide, and to compositions and methods of making and using such complexes. In the compositions provided herein, one or more of the minerals, mineral-amino acid compounds, or mineral-amino acid/polysaccharide complexes have a coating that can delay release of the mineral or chelated (or partially chelated) mineral until the it reaches the small intestine, which can reduce the likelihood of stomach upset.

### BACKGROUND

Minerals are required as part of the human diet for good health. For example, calcium is a major component of bones and teeth, iron is an essential constituent of hemoglobin, copper, magnesium, and zinc are co-factors for a variety of enzymes, and manganese and selenium can function as antioxidants and contribute to endothelial integrity. Mineral deficiencies can lead to poor health and specific disorders. The human body requires traces of certain minerals (e.g., calcium, iron, copper, and zinc) in soluble form to provide metal ions that are bioavailable within the bloodstream. With the high consumption of processed and convenience foods, however, there are concerns that the typical present day diet may not contain sufficient levels of such minerals.

Naturally occurring minerals found in foods are often chelated or bound within an organic matrix. In contrast, minerals found in dietary supplements typically are in the form of an inorganic salt, such as a mineral sulfate. These inorganic mineral species are more reactive and can catalyze production of free radicals, which have been associated with various degenerative diseases or conditions within the digestive tract. In addition, certain mineral species may cause stomach upset, and may also have relatively low bioavailability.

### SUMMARY

This document provides complexes containing minerals, mineral-amino acid compounds, and/or complexes containing mineral-amino acid compounds coupled to polysaccharides, as well as compositions containing the complexes, and methods for making and using the complexes and compositions. At least some of the minerals, mineral-amino acid compounds, or mineral-amino acid/polysaccharide complexes in the compositions provided herein are coated to slow or delay release of the mineral component (e.g., until the complexes have entered the small intestine), in order to reduce the likelihood that the complexes will cause stomach upset.

In a first aspect, this document features a composition. The composition can contain, consist essentially of, or consist of a first complex that includes a first compound conjugated to a first polysaccharide, where the first compound includes a first mineral and a first amino acid, and where the first complex is coated with a slow release coating. The first mineral can be selected from the group consisting of zinc, copper, selenium, manganese, chromium, and iron. The first amino acid can be selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. The first amino acid can be selected from the group consisting of L-glycine and L-aspartic acid. The first polysaccharide can be selected from the group consisting of cellulose, polyhexoses, polypentoses, polydextrose, starch, polygalactan, polymannan, chitin, chitosan, chondroitin, polyfructose, inulin, pectin, and derivatives thereof. The first polysaccharide can be selected from the group consisting of a cellulose derivative, polyfructose, inulin, and polydextrose. The slow release coating can include sodium alginate, stearic acid, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, monoglycerides, diglycerides, or any combination thereof. The first complex can be selected from the group consisting of a zinc-amino acid/polyfructose complex, a copper-amino acid/polyfructose complex, a selenium-amino acid/polyfructose complex, a manganese-amino acid/polyfructose complex, a chromium-amino acid/polyfructose complex, and an iron-amino acid/polyfructose complex. The composition can further include one or more of vitamin A, vitamin C, vitamin D, vitamin E, vitamin K, thiamin, riboflavin, niacin, vitamin B6, folate, vitamin B12, biotin, pantothenic acid, and phosphorous.

In some cases, the composition can further include a second complex that includes a second compound conjugated to a second polysaccharide, where the second compound includes a second mineral and a second amino acid, where the second mineral is different from the first mineral, and where the second complex is not coated with a slow release coating. The second mineral can be selected from the group consisting of magnesium, calcium, boron, iodine, potassium, and molybdenum. The first amino acid and the second amino acid can be selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. The first amino acid and the second amino acid can be selected from the group consisting of L-glycine and L-aspartic acid. The first amino acid and the second amino acid can be the same, or the first amino acid and the second amino acid can be different. The first polysaccharide and the second polysaccharide can be selected from the group consisting of cellulose, polyhexoses, polypentoses, polydextrose, starch, polygalactan, polymannan, chitin, chitosan, chondroitin, polyfructose, inulin, pectin, and derivatives thereof. The first polysaccharide and the second polysaccharide can be the same, or the first polysaccharide and the second polysaccharide can be different. The first polysaccharide and the second polysaccharide can be selected from the group consisting of a cellulose derivative, polyfructose, inulin, and polydextrose. The first complex can be selected from the group consisting of a zinc-amino acid/polyfructose complex, a copper-amino acid/polyfructose complex, a selenium-amino acid/polyfructose complex, a manganese-amino acid/polyfructose complex, a chromium-amino acid/polyfructose complex, and an iron-amino acid/polyfructose complex, and the second complex can be selected from the group consisting of a calcium-amino acid/polyfructose complex, an iodine-amino acid/polyfructose complex, a magnesium-amino acid/polyfructose complex, a potassium-amino acid/polyfructose complex, a molybdenum-amino acid/polyfructose complex, and a boron-amino acid/polyfructose complex. The composition can further include one or more of vitamin A, vitamin C, vitamin D, vitamin E, vitamin K, thiamin, riboflavin, niacin, vitamin B6, folate, vitamin B12, biotin, pantothenic acid, and phosphorous.

In another aspect, this document features a method for preparing a coated mineral-amino acid/polysaccharide complex. The method can include, or consist essentially of, dry-blending a combination of (a) one or more mineral-amino acid compounds having a particle size smaller than about 80 mesh with (b) a polysaccharide and (c) a slow release coating material; granulating the dry-blended combination to generate a granulated material; and drying the granulated material. The drying can include using a fluid bed dryer or a drying room. The method can further include wet milling the granulated material to reduce the particle size of the material to 10 mesh or smaller, in which case the drying step can include drying the wet milled granulated material.

In another aspect, this document features a composition containing, consisting essentially of, or consisting of a first compound that includes a first mineral coupled to a first amino acid, where the first compound is coated with a slow release coating. The first mineral can be selected from the group consisting of zinc, copper, selenium, manganese, chromium, and iron. The first amino acid can be selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. The first amino acid can be selected from the group consisting of L-glycine and L-aspartic acid. The slow release coating can include sodium alginate, stearic acid, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, monoglycerides, diglycerides, or any combination thereof. The first compound can be selected from the group consisting of a zinc-amino acid, a copper-amino acid, a selenium-amino acid, a manganese-amino acid, a chromium-amino acid, and an iron-amino acid. The composition can further contain one or more of vitamin A, vitamin C, vitamin D, vitamin E, vitamin K, thiamin, riboflavin, niacin, vitamin B6, folate, vitamin B12, biotin, pantothenic acid, and phosphorous.

In some cases, the composition can further include a second compound that contain a second mineral coupled to a second amino acid, where the second mineral is different from the first mineral, and where the second compound is not coated with a slow release coating. The second mineral can be selected from the group consisting of magnesium, calcium, boron, iodine, potassium, and molybdenum. The first amino acid and the second amino acid can be selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. The first amino acid and the second amino acid can be selected from the group consisting of L-glycine and L-aspartic acid. The first amino acid and the second amino acid can be the same, or the first amino acid and the second amino acid can be different. The first compound can be selected from the group consisting of a zinc-amino acid, a copper-amino acid, a selenium-amino acid, a manganese-amino acid, a chromium-amino acid, and an iron-amino acid, and the second compound can be selected from the group consisting of a calcium-amino acid, an iodine-amino acid, a magnesium-amino acid, a potassium-amino acid, a molybdenum-amino acid, and a boron-amino acid. The composition can further contain one or more of vitamin A, vitamin C, vitamin D, vitamin E, vitamin K, thiamin, riboflavin, niacin, vitamin B6, folate, vitamin B12, biotin, pantothenic acid, and phosphorous.

In still another aspect, this document features a method for making a coated mineral-amino acid compound. The method can include, or consist essentially of, dry-blending a combination of (a) one or more mineral-amino acid compounds having a particle size smaller than about 80 mesh with (b) a slow release coating material; granulating the dry-blended combination to generate a granulated material; and drying the granulated material. The drying can include using a fluid bed dryer or a drying room. The method can further include wet milling the granulated material to reduce the particle size of the material to 10 mesh or smaller.

In another aspect, this document features a method for making a coated mineral-amino acid compound, wherein the method includes, or consists essentially of, dry-blending one or more mineral-amino acid compounds having a particle size smaller than about 80 mesh in a fluid bed, granulating the dry-blended compounds in the fluid bed, and adding a slow release coating material to the mineral-amino acid compounds during the dry-blending step, during the granulating step, or during the dry-blending step and the granulating step. The method can further include drying the granulated material. The fluid bed can be in a top spray configuration or a bottom spray configuration.

This document also features a composition comprising, consisting essentially of, or consisting of particles of one or more minerals or mineral salts, where the particles are coated with a slow release coating. The one or more minerals or mineral salts can include one or more of zinc, copper, selenium, manganese, chromium, iron, a zinc salt, a copper salt, a selenium salt, a manganese salt, a chromium salt, and an iron salt. The composition can further include particles of one or more minerals or mineral salts that are not coated with a slow release coating. The particles that are not coated can contain one or more of magnesium, calcium, boron, iodine, potassium, molybdenum, sodium, a magnesium salt, a calcium salt, a boron salt, an iodine salt, a potassium salt, a molybdenum salt, and a sodium salt. In some cases, the composition can include a chromium salt, a copper salt, a manganese salt, a zinc salt, an iron salt, a sodium salt, a magnesium salt, and a calcium salt. The slow release coating can include sodium alginate, stearic acid, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, monoglycerides, diglycerides, or any combination thereof.

As used herein, the term “about” when used to refer to weight % in a composition means±10% of the reported weight %. As used herein, the term “about” when used to refer to measured characteristics of the composition means±20% of the reported value. As used herein, the term “about” when used to a condition for making the composition means±20% of the value.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 includes a pair of images showing magnesium powder before (left) and after (right) granulation and spray coating processes.

FIG. 2 includes a pair of images showing spray coated (left) and uncoated (right) magnesium mixed with water. The granulated and spray coated magnesium displayed delayed dissolution in water when compared to the uncoated magnesium.

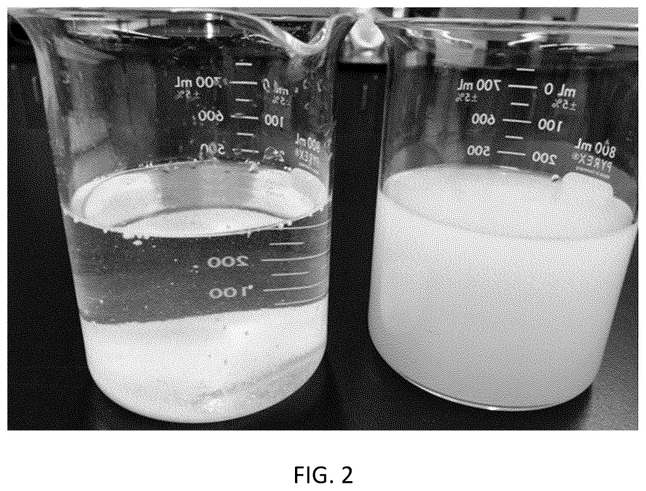

### DETAILED DESCRIPTION

In some cases, this document provides complexes that include a mineral-amino acid compound, optionally conjugated to a polysaccharide, as well as compositions containing two or more of the mineral-amino acid complexes or mineral-amino acid/polysaccharide complexes, methods for making the complexes and compositions, and methods for using the complexes and compositions. The mineral-amino acid compound and the polysaccharide in the mineral-amino acid/polysaccharide complexes provided herein can be conjugated to one another by any appropriate interaction. For example, a mineral-amino acid compound can be conjugated to a polysaccharide by one or more of a covalent bond, a coordinate covalent bond, Van der Waals interactions, a hydrophobic bond, a hydrogen bond, or an ionic bond.

The mineral-amino acid compounds in the complexes and compositions provided herein can include any mineral having a dietary or health benefit that is chelated, is partially chelated, or complexed (e.g., forms a salt with) an amino acid. In some cases, the mineral-amino acid compound can be an amino acid-chelated mineral. For example, the mineral-amino acid compound can be a mineral-amino acid complex or salt. Examples of minerals that can be included in the complexes include, without limitation, chromium, calcium, copper, iron, magnesium, manganese, molybdenum, potassium, zinc, selenium, iodine, and any combination thereof.

Any amino acid that can form a chelate or complex with a mineral can be used in the complexes and compositions provided herein. In some cases, for example, the amino acid portion of a mineral-amino acid compound can include one or more natural or unnatural amino acids. As used herein, the term “natural” amino acid refers to one of the twenty commonly occurring amino acids. Natural amino acids can be in their D or L form. For example, a natural amino acid can be selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, L-valine, and combinations thereof. In some cases, for example, the amino acid can be L-glycine or L-aspartic acid.

When present, the polysaccharides in the complexes and compositions provided herein can include any suitable grouping of non-hydrolysable sugars (monosaccharides) linked together. Non-limiting examples of suitable polysaccharides include cellulose derivatives, polyhexoses and derivatives thereof, and polypentoses and derivatives thereof. In some cases, a complex can include one or more polysaccharides be selected from polydextrose, starch, polygalactan, polymannan, chitin, chitosan, chondroitin, polyfructose, pectin, fructooligosaccharide, and derivatives thereof. In some cases, a polysaccharide can be a polyfructose (e.g., inulin). When included in a complex provided herein, the inulin can have a degree of polymerization ranging from about 2 to about 100 (e.g., about 2 to 10, about 12 to 15, about 20 to 30, about 25 to 45, about 30 to 40, about 50 to 75, about 45 to 65, about 50 to 55, about 70 to 80, about 75 to 90, or about 92 to 100).

The compositions provided herein that contain mineral-amino acid/polysaccharide complexes can contain a combination of different mineral-amino acid compound/polysaccharide complexes, where each of the mineral-amino acid/polysaccharide complexes includes one or more mineral-amino acid compounds conjugated to one or more polysaccharides. For example, a composition can include a first complex that contains a first mineral-first amino acid compound conjugated to a first polysaccharide, and a second complex that contains a second mineral-second amino acid compound conjugated to a second polysaccharide. It is to be noted that various combinations of minerals, amino acids and polysaccharides can be used in the different complexes, and that the same type of amino acid and/or polysaccharide can be combined with different minerals.

Thus, in some cases, the compositions provided herein contain one or more (e.g., two or more, three or more, four or more, five or more, or six or more) mineral-amino acid/polysaccharide complexes. For example, a composition can contain two or more of a calcium-amino acid polysaccharide complex, an iron-amino acid polysaccharide complex, an iodine-amino acid polysaccharide complex, a magnesium-amino acid polysaccharide complex, a zinc-amino acid polysaccharide complex, a selenium-amino acid polysaccharide complex, a copper-amino acid polysaccharide complex, a manganese-amino acid polysaccharide complex, a molybdenum-amino acid polysaccharide complex, a chromium-amino acid polysaccharide complex, and a boron-amino acid polysaccharide complex. In some cases, a mineral-amino acid polysaccharide complex can include a mineral-amino acid compound containing 75% aspartate and 25% glycinate. In some cases, a mineral-amino acid polysaccharide complex can include polyfructose. In some cases, the mineral-amino acid polysaccharide complex can include inulin having a degree of polymerization ranging from about 2 to about 100. For example, in some cases, a mineral-amino acid polysaccharide complex can include inulin having a degree of polymerization of about 12-15.

The compositions provided herein that contain mineral-amino acid compounds can contain a combination of different mineral-amino acid compounds, where each of the mineral-amino acid compounds includes one or more minerals coupled to one or more amino acids. For example, a composition can include a first compound that contains a first mineral coupled to a first amino acid, and a second compound that contains a second mineral coupled to a second amino acid. It is to be noted that various combinations of minerals and amino acids can be used in the different compounds, and that the same type of amino acid can be combined with different minerals or the same type of mineral can be combined with different amino acids.

Thus, in some cases, the compositions provided herein contain one or more (e.g., two or more, three or more, four or more, five or more, or six or more) mineral-amino acid compounds. For example, a composition can contain two or more of a calcium-amino acid compound, an iron-amino acid compound, an iodine-amino acid compound, a magnesium-amino acid compound, a zinc-amino acid compound, a selenium-amino acid compound, a copper-amino acid compound, a manganese-amino acid compound, a molybdenum-amino acid compound, a chromium-amino acid compound, and a boron-amino acid compound. In some cases, a mineral-amino acid compound can include a mineral-amino acid compound containing about 75% aspartate and about 25% glycinate.

In some cases, this document provides compositions that contain mineral or mineral salts that are not chelated to an amino acid and are not complexed to a polysaccharide. Any mineral having a dietary or health benefit can be included. Non-limiting examples of non-chelated, uncomplexed minerals and mineral salts that can be used include calcium carbonate, calcium citrate, calcium gluconate, calcium lactate, magnesium carbonate, magnesium citrate, magnesium lactate, chromium chloride, copper sulfate, manganese sulfate, sodium selenite, zinc sulfate, ferrous sulfate, and any combination thereof.

In some cases, the compositions provided herein can consist of or consist essentially of one or more listed ingredients. By “consisting essentially of” with regard to a composition is meant that the composition contains specified ingredients, and can contain additional ingredients that do not materially affect the basic and novel characteristics of the composition.

Within each compositions provided herein, at least one of the minerals or mineral salts, mineral-amino acid compounds, or mineral-amino acid/polysaccharide complexes has a slow release coating that can control and/or delay mineral release from the complex, thus preventing or delaying dissolution or disintegration of the at least one mineral, compound, or complex in the stomach. In some cases, at least one other mineral or mineral salt, mineral-amino acid compound, or mineral-amino acid/polysaccharide complex in the same composition lacks a slow release coating. Including a slow release coating on a mineral or mineral salt, mineral-amino acid complex, or mineral-amino acid/polysaccharide complex can reduce the likelihood that the mineral, mineral salt, mineral-amino acid compound, or mineral-amino acid/polysaccharide complex will cause stomach upset after administration to a mammal. This can be useful because some minerals are more likely than others to cause stomach upset. For example, zinc, copper, selenium, manganese, chromium, and iron are more likely than some other minerals to cause an upset stomach after ingestion. Thus, including a slow release coating on compounds or complexes that contain one or more of zinc, copper, selenium, manganese, chromium, and iron can reduce the likelihood that the complexes will cause stomach upset. Complexes that do not have a coating can contain other minerals, such as magnesium, calcium, boron, iodine, potassium, and/or molybdenum. Similarly, including a slow release coating on minerals or mineral salts that include one or more of zinc, copper, selenium, manganese, chromium, and iron can reduce the likelihood that the minerals or mineral salts will cause stomach upset. Other minerals or mineral salts that do not have a coating also can be included, such as magnesium, calcium, boron, iodine, potassium, molybdenum, sodium, salts thereof, and any combination thereof. Thus, in some cases, a composition provided herein can include one or more coated minerals selected from zinc, copper, selenium, manganese, chromium, iron, and salts thereof (e.g., chromium, copper, manganese, zinc, and iron, or salts thereof), and one or more uncoated minerals selected from magnesium, calcium, boron, iodine, potassium, molybdenum, sodium, and salts thereof (e.g., sodium, magnesium, and calcium, or salts thereof).

Any appropriate material can be used in a coating. In some cases, for example, a complex described herein can have a coating that includes, without limitation, stearic acid, sodium alginate, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, mono and/or diglycerides, or any combination thereof.

The compositions provided herein can contain one or more coated mineral-amino acid complexes or mineral-amino acid/polysaccharide complexes. The coated complexes in a composition can include zinc-containing complexes, copper-containing complexes, selenium-containing complexes, manganese-containing complexes, chromium-containing complexes, iron-containing complexes, or any combination thereof (e.g., any two, three, four, five, or all six of zinc-, copper-, selenium-, manganese-, chromium-, and iron-containing complexes). For example, a composition can contain a combination of coated complexes that includes zinc-containing complexes and copper-containing complexes; zinc-containing complexes and selenium-containing complexes; zinc-containing complexes and manganese-containing complexes; zinc-containing complexes and chromium-containing complexes; zinc-containing complexes and iron-containing complexes; copper-containing complexes and selenium-containing complexes; copper-containing complexes and manganese-containing complexes; copper-containing complexes and chromium-containing complexes; copper-containing complexes and iron-containing complexes; selenium-containing complexes and manganese-containing complexes; selenium-containing complexes and chromium-containing complexes; selenium-containing complexes and iron-containing complexes; manganese-containing complexes and chromium-containing complexes; manganese-containing complexes and iron-containing complexes; chromium-containing complexes and iron-containing complexes; zinc-containing complexes, copper-containing complexes, and selenium-containing complexes; zinc-containing complexes, copper-containing complexes, and manganese-containing complexes; zinc-containing complexes, copper-containing complexes, and chromium-containing complexes; zinc-containing complexes, copper-containing complexes, and iron-containing complexes; zinc-containing complexes, selenium-containing complexes, and manganese-containing complexes; zinc-containing complexes, selenium-containing complexes, and chromium-containing complexes; zinc-containing complexes, selenium-containing complexes, and iron-containing complexes; zinc-containing complexes, manganese-containing complexes, and chromium-containing complexes; zinc-containing complexes, manganese-containing complexes, and iron-containing complexes; zinc-containing complexes, chromium-containing complexes, and iron-containing complexes; copper-containing complexes, selenium-containing complexes, and manganese-containing complexes; copper-containing complexes, selenium-containing complexes, and chromium-containing complexes; copper-containing complexes, selenium-containing complexes, and iron-containing complexes; copper-containing complexes, manganese-containing complexes, and chromium-containing complexes; copper-containing complexes, manganese-containing complexes, and iron-containing complexes; copper-containing complexes, chromium-containing complexes, and iron-containing complexes; selenium-containing complexes, manganese-containing complexes, and chromium-containing complexes; selenium-containing complexes, manganese-containing complexes, and iron-containing complexes; selenium-containing complexes, chromium-containing complexes, and iron-containing complexes; manganese-containing complexes, chromium-containing complexes, and iron-containing complexes; zinc-containing compounds, copper-containing compounds, selenium-containing compounds, and manganese-containing compounds; zinc-containing compounds, copper-containing compounds, selenium-containing compounds, and chromium-containing compounds; zinc-containing compounds, copper-containing compounds, selenium-containing compounds, and iron-containing compounds; zinc-containing compounds, copper-containing compounds, manganese-containing compounds, and chromium-containing compounds; zinc-containing compounds, copper-containing compounds, manganese-containing compounds, and iron-containing compounds; zinc-containing compounds, copper-containing compounds, chromium-containing compounds, and iron-containing compounds; zinc-containing compounds, selenium-containing compounds, manganese-containing compounds, and chromium-containing compounds; zinc-containing compounds, selenium-containing compounds, manganese-containing compounds, and iron-containing compounds; zinc-containing compounds, selenium-containing compounds, chromium-containing compounds, and iron-containing compounds; zinc-containing compounds, manganese-containing compounds, chromium-containing compounds, and iron-containing compounds; copper-containing compounds, selenium-containing compounds, manganese-containing compounds, and chromium-containing compounds; copper-containing compounds, selenium-containing compounds, manganese-containing compounds, and iron-containing compounds; copper-containing compounds, selenium-containing compounds, chromium-containing compounds, and iron-containing compounds; copper-containing compounds, manganese-containing compounds, chromium-containing compounds, and iron-containing compounds; selenium-containing compounds, manganese-containing compounds, chromium-containing compounds, and iron-containing compounds; zinc-containing compounds, copper-containing compounds, selenium-containing compounds, manganese-containing compounds, chromium-containing compounds; zinc-containing compounds, copper-containing compounds, selenium-containing compounds, manganese-containing compounds, and iron-containing compounds; zinc-containing compounds, copper-containing compounds, selenium-containing compounds, chromium-containing compounds, and iron-containing compounds; zinc-containing compounds, copper-containing compounds, manganese-containing compounds, chromium-containing compounds, and iron-containing compounds; zinc-containing compounds, selenium-containing compounds, manganese-containing compounds, chromium-containing compounds, and iron-containing compounds; copper-containing compounds, selenium-containing compounds, manganese-containing compounds, chromium-containing compounds, and iron-containing compounds; or zinc-containing complexes, copper-containing compounds, selenium-containing compounds, manganese-containing compounds, chromium-containing compounds, and iron-containing compounds.

The mineral-amino acid/polysaccharide complexes in the compositions provided herein can contain any appropriate ratio of mineral-amino acid compound to polysaccharide. For example, the ratio (by weight) of mineral-amino acid compound to polysaccharide in the complexes can range from 10:1 to 1:10 (e.g., 10:1, 9:1, 8:1, 7:1, 6:1; 5:1; 4:1; 3:1; 2:1; 1.5:1; 1:1, 1:1.5, 1:2, 1:3, 1:4, 1:5, 1:6, 1:7, 1:8, 1:9, or 1:10). In some cases, a complex can have a ratio of mineral-amino acid compound to polysaccharide of 5:1, or a ratio of mineral-amino acid compound to polysaccharide of 1:1. In some cases, a mineral-amino acid/polysaccharide complex can be zinc aspartate:zinc glycinate:inulin (degree of polymerization of 12-15) having a ratio of 50:30:20 by weight. In some cases, a mineral-amino acid polysaccharide complex can be iron aspartate:iron glycinate:inulin (degree of polymerization of 2-10) having a ratio of 25:25:50 by weight.

The coated complex(es) in a composition can contain any appropriate amount of coating, any appropriate ratio of coating to complex, and any appropriate ratio of coating to mineral-amino acid compound to polysaccharide. For example, the amount of coating in a coated complex can range from about 1% to about 70% by weight (e.g., about 1 to 3% by weight, about 3 to 5% by weight, about 5 to 10% by weight, about 10 to 20% by weight, about 20 to 30% by weight, about 30 to 40% by weight, about 40 to 50% by weight, about 50 to 60% by weight, or about 60 to 70% by weight). The ratio of coating to mineral-amino acid compound to polysaccharide can range from 1:100 to 7:10 (e.g., 1:100, 1:50, 3:100, 1:25, 1:20, 3:50, 7:100, 2:25, 9:100, 1:10, 1:5, 3:10, 2:5, 1:2, 3:5, or 7:10). In some cases, for example, a coated complex can include a ratio of chelated or semi-chelated mineral:polysaccharide: coating, by weight, of 6:1:1.

The complexes described herein can have properties that distinguish them from a simple mixture of the same mineral-amino acid compound and polysaccharide. For example, a complex of a mineral-amino acid compound and a polysaccharide can diffuse more slowly across a 3500 MW cutoff dialysis membrane than an uncomplexed mixture of the same mineral-amino acid compound and polysaccharide. In addition, a mineral-amino acid/polysaccharide complex can exhibit a different Fourier-Transform Near-IR (FT-NIR) spectrum than an uncomplexed mixture of the same mineral-amino acid compound and polysaccharide, as determined by an industry standard correlation factor.

In some cases, the compositions provided herein can include one or more ingredients in addition to the mineral-amino acid complexes or mineral-amino acid/polysaccharide complexes. For example, a composition can include one or more of vitamin A, vitamin C, vitamin D, vitamin E, vitamin K, thiamin, riboflavin, niacin, vitamin B6, folate, vitamin B12, biotin, pantothenic acid, and phosphorous, with or without other botanical and/or dietary ingredients (e.g., fish oil, phosphatidylserine, Echinacea purpurea, Astragalus membranaceus, Aronia melanocapa, resveratrol, epigallocatechin gallate, Curcuma longa, Bacopa moniera, grape extracts, olive extracts, bilberry (Vaccinium myrtilus), Ginko biloba, or herbs with polyphenols and flavonoids).

The phrase “total daily dose” as used herein refers to the amount of active ingredient administered over a 24 hour period. For example, the amount of zinc amino acid polysaccharide complex in a total daily dose is calculated based on the amount of zinc administered over 24 hours, not on the amount of zinc amino acid polysaccharide complex administered over 24 hours. A total daily dose can be prepared for administration in the form of one or more dosage units (e.g., two, three, four, five, or six tablets, capsules, gelcaps, sofgels, or gummies). In some cases, the one or more dosage units can be administered in one or more dosages over the course of 24 hours (e.g., one dose, two doses, three doses, four doses, five doses, or six doses), where the total amount of active ingredient in the one or more dosages does not exceed the total daily dose.

In some cases, the compositions provided herein can be formulated for oral administration and can include suitable excipients, flavorings, colorants, and other ingredients. For oral administration, tablets, capsules, gelcaps, sofgels, or gummies can be prepared with one or more pharmaceutically acceptable excipients or carriers, such as a binding agent, filler, lubricant, disintegrant, or wetting agent. In some cases, for example, a composition provided herein can contain a pharmaceutically acceptable carrier for administration to a mammal.

In some cases, a composition provided herein can contain pantothenic acid (e.g., as calcium pantothenate). The pantothenic acid can be included in any appropriate amount. For example, a composition can contain pantothenic acid in an amount sufficient to provide a daily dose from about 10 mg to about 200 mg (e.g., about 10 to about 25 mg, about 25 to about 50 mg, about 50 to about 100 mg, about 100 to about 150 mg, or about 150 to about 200 mg).

In some cases, a composition provided herein can contain calcium (e.g., as calcium carbonate, dicalcium phosphate, calcium citrate, calcium gluconate, calcium amino acid polysaccharide complex). The calcium can be included in any appropriate amount. For example, a composition can contain calcium in an amount sufficient to provide a daily dose from about 200 mg to about 1000 mg (e.g., about 200 to about 250 mg, about 250 to about 500 mg, about 500 to about 750 mg, or about 750 to about 1000 mg).

In some cases, a composition provided herein can contain iron (e.g., as an iron amino acid polysaccharide complex). The iron can be included in any appropriate amount. For example, a composition can contain iron in an amount sufficient to provide a daily dose from about 0.01 mg to about 18 mg (e.g., about 0.01 to about 0.1 mg, about 0.1 to about 0.5 mg, about 0.5 to about 1 mg, about 1 to about 3 mg, about 3 to about 5 mg, about 5 to about 10 mg, about 10 to about 15 mg, or about 15 to about 18 mg).

In some cases, a composition provided herein can contain phosphorus (e.g., as dicalcium phosphate). The phosphorus can be included in any appropriate amount. For example, a composition can contain phosphorus in an amount sufficient to provide a daily dose from about 0.01 mg to about 300 mg (e.g., about 0.01 to about 0.1 mg, about 0.1 to about 1 mg, about 1 to about 10 mg, about 10 to about 50 mg, about 50 to about 100 mg, about 100 to about 200 mg, or about 200 to about 300 mg).

In some cases, a composition provided herein can contain iodine (e.g., as iodine amino acid polysaccharide complex). The iodine can be included in any appropriate amount. For example, a composition can contain iodine in an amount sufficient to provide a daily dose from about 100 μg to about 300 μg (e.g., about 100 to about 150 μg, about 150 to about 200 μg, about 200 to about 250 μg, or about 250 to about 300 μg).

In some cases, a composition provided herein can contain magnesium (e.g., as magnesium oxide, magnesium lactate, magnesium citrate, magnesium amino acid polysaccharide complex). The magnesium can be included in any appropriate amount. For example, a composition can contain magnesium in an amount sufficient to provide a daily dose from about 100 to about 400 mg (e.g., about 100 to about 150 mg, about 150 to about 200 mg, about 200 to about 250 mg, about 250 to about 300 mg, or about 300 to about 400 mg).

In some cases, a composition provided herein can contain zinc (e.g., as a zinc amino acid polysaccharide complex). The zinc can be included in any appropriate amount. For example, a composition can contain zinc in an amount sufficient to provide a daily dose from about 5 mg to about 30 mg (e.g., about 5 to about 10 mg, about 10 to about 15 mg, about 15 to about 20 mg, about 20 to about 25 mg, or about 25 to about 30 mg).

In some cases, a composition provided herein can contain selenium (e.g., as a selenium amino acid polysaccharide complex). The selenium can be included in any appropriate amount. For example, a composition can contain selenium in an amount sufficient to provide a daily dose from about 35 μg to about 150 μg (e.g., about 35 to about 50 μg, about 50 to about 75 μg, about 75 to about 100 ag, about 100 to about 125 ag, or about 125 to about 150 μg).

In some cases, a composition provided herein can contain copper (e.g., as a copper amino acid polysaccharide complex). The copper can be included in any appropriate amount. For example, a composition can contain copper in an amount sufficient to provide a daily dose from about 1 mg to about 5 mg (e.g., about 1 to about 2 mg, about 2 to about 3 mg, about 3 to about 4 mg, or about 4 to about 5 mg).

In some cases, a composition provided herein can contain manganese (e.g., as a manganese amino acid polysaccharide complex). The manganese can be included in any appropriate amount. For example, a composition can contain manganese in an amount sufficient to provide a daily dose from about 1 mg to about 5 mg (e.g., about 1 to about 2 mg, about 2 to about 3 mg, about 3 to about 4 mg, or about 4 to about 5 mg).

In some cases, a composition provided herein can contain chromium (e.g., as a chromium amino acid polysaccharide complex). The chromium can be included in any appropriate amount. For example, a composition can contain chromium in an amount sufficient to provide a daily dose from about 60 μg to about 360 μg (e.g., about 60 to about 100 μg, about 100 to about 150 μg, about 150 to about 200 μg, about 200 to about 250 μg, about 250 to about 300 μg, or about 300 to about 360 μg).

In some cases, a composition provided herein can contain molybdenum (e.g., as a molybdenum amino acid polysaccharide complex). The molybdenum can be included in any appropriate amount. For example, a composition can contain molybdenum in an amount sufficient to provide a daily dose from about 50 μg to about 150 μg (e.g., about 50 to about 75 μg, about 75 to about 100 μg, about 100 to about 125 μg, or about 125 to about 150 μg).

In some cases, a composition provided herein can contain boron (e.g., as a boron amino acid polysaccharide complex). The boron can be included in any appropriate amount. For example, a composition can contain boron in an amount sufficient to provide a daily dose from about 0.01 μg to about 300 μg (e.g., about 0.01 to about 0.1 ag, about 0.1 to about 1 ag, about 1 to about 10 ag, about 10 to about 50 ag, about 50 to about 100 μg, about 100 to about 200 ag, or about 200 to about 300 ag).

In some cases, a composition provided herein can contain vitamin A (e.g., as beta carotene). The vitamin A can be included in any appropriate amount. For example, a composition can contain vitamin A in an amount sufficient to provide a daily dose from about 1 IU to about 5000 IU (e.g., about 1 to about 10 IU, about 10 to about 100 IU, about 100 to about 500 IU, about 500 to about 1000 IU, about 1000 to about 2500 IU, or about 2500 to about 5000 IU).

In some cases, a composition provided herein can contain vitamin C (e.g., as ascorbic acid). The vitamin C can be included in any appropriate amount. For example, a composition can contain vitamin C in an amount sufficient to provide a daily dose from about 5 mg to about 1000 mg (e.g., about 5 to about 10 mg, about 10 to about 50 mg, about 50 to about 100 mg, about 100 to about 250 mg, about 250 to about 500 mg, or about 500 to about 1000 mg).

In some cases, a composition provided herein can contain vitamin D (e.g., as cholecalciferol). The vitamin D can be included in any appropriate amount. For example, a composition can contain vitamin D in an amount sufficient to provide a daily dose from about 1 IU to about 4000 IU (e.g., about 1 to about 10 IU, about 10 to about 100 IU, about 100 to about 500 IU, about 500 to about 1000 IU, about 1000 to about 2000 IU, about 2000 to about 3000 IU, or about 3000 to about 4000 IU).

In some cases, a composition provided herein can contain vitamin E (e.g., as d-alpha tocopheryl succinate). The vitamin E can be included in any appropriate amount. For example, a composition can contain vitamin E in an amount sufficient to provide a daily dose from about 5 IU to about 60 IU (e.g., about 5 to about 10 IU, about 10 to about 20 IU, about 20 to about 30 IU, about 30 to about 40 IU, about 40 to about 50 IU, or about 50 to about 60 IU).

In some cases, a composition provided herein can contain vitamin K (e.g., as phytonadione). The vitamin K can be included in any appropriate amount. For example, a composition can contain vitamin K in an amount sufficient to provide a daily dose from about 0.01 μg to about 56 μg (e.g., about 0.01 to about 0.1 μg, about 0.1 to about 1 μg, about 1 to about 10 μg, about 10 to about 20 μg, about 20 to about 30 μg, about 30 to about 40 μg, about 40 to about 50 μg, or about 50 to about 56 μg).

In some cases, a composition provided herein can contain thiamin (e.g., as thiamin HCl). The thiamin can be included in any appropriate amount. For example, a composition can contain thiamin in an amount sufficient to provide a daily dose from about 1.5 mg to about 15 mg (e.g., about 1.5 to about 2.5 mg, about 2.5 to about 5 mg, about 5 to about 8 mg, about 8 to about 10 mg, about 10 to about 12 mg, or about 12 to about 15 mg).

In some cases, a composition provided herein can contain riboflavin. The riboflavin can be included in any appropriate amount. For example, a composition can contain riboflavin in an amount sufficient to provide a daily dose from about 1.7 mg to about 17 mg (e.g., about 1.7 to about 2.5 mg, about 2.5 to about 5 mg, about 5 to about 7.5 mg, about 7.5 to about 10 mg, about 10 to about 12.5 mg, or about 12.5 to about 15 mg).

In some cases, a composition provided herein can contain niacin (e.g., as niacinamide). The niacin can be included in any appropriate amount. For example, a composition can contain niacin in an amount sufficient to provide a daily dose from about 2 mg to about 100 mg (e.g., about 2 to about 10 mg, about 10 to about 25 mg, about 25 to about 50 mg, about 50 to about 100 mg, about 100 to about 150 mg, or about 150 to about 200 mg).

In some cases, a composition provided herein can contain vitamin B6 (e.g., as pyridoxine HCl). The vitamin B6 can be included in any appropriate amount. For example, a composition can contain vitamin B6 in an amount sufficient to provide a daily dose from about 2 mg to about 20 mg (e.g., about 2 to about 5 mg, about 5 to about 8 mg, about 8 to about 10 mg, about 10 to about 15 mg, or about 15 to about 20 mg).

In some cases, a composition provided herein can contain folate (e.g., as folic acid or methyl folate). The folate can be included in any appropriate amount. For example, a composition can contain folate in an amount sufficient to provide a daily dose from about 200 μg to about 1200 μg (e.g., about 200 to about 400 μg, about 400 to about 600 μg, about 600 to about 800 μg, about 800 to about 1000 μg, or about 1000 to about 1200 μg).

In some cases, a composition provided herein can contain vitamin B12 (e.g., as cyanocobalamin or methylcobalamin). The vitamin B12 can be included in any appropriate amount. For example, a composition can contain vitamin B12 in an amount sufficient to provide a daily dose from about 6 μg to about 18 μg (e.g., about 6 to about 8 ag, about 8 to about 10 μg, about 10 to about 12 ag, about 12 to about 14 ag, about 14 to about 16 μg, or about 16 to about 18 μg).

In some cases, a composition provided herein can contain biotin (e.g., as d biotin). The biotin can be included in any appropriate amount. For example, a composition can contain biotin in an amount sufficient to provide a daily dose from about 20 μg to about 400 μg (e.g., about 20 to about 50 ag, about 50 to about 100 ag, about 100 to about 200 μg, about 200 to about 300 μg, or about 300 to about 400 μg).

In some cases, a composition can contain ingredients (not including excipients, carriers, or coatings) as listed in TABLE 1, or as listed in any of TABLES 2-12.

TABLE 1

| | Total Daily | |
|---|---|---|
| | Dose | |
| Vitamin A (as beta carotene) | 1-5000 | IU |
| Vitamin C (as ascorbic acid) | 5-1000 | mg |
| Vitamin D (as cholecalciferol) | 1-4000 | IU |
| Vitamin E (as d-alpha tocopheryl succinate) | 5-60 | IU |
| Vitamin K (as phytonadione) | 0-56 | μg |
| Thiamin (as thiamin HCl) | 1.5-15 | mg |
| Riboflavin | 1.7-17 | mg |
| Niacin (as niacinamide) | 2-100 | mg |
| Vitamin B6 (as pyridoxine HCl) | 2-20 | mg |
| Folate (as folic acid or the methylated form) | 200-1200 | μg |
| Vitamin B12 (as cyanocobalamin or methylcobalamin) | 6-18 | μg |
| Biotin (as d-biotin) | 20-400 | μg |
| Pantothenic Acid (as calcium pantothenate) | 10-200 | mg |
| Calcium (as calcium carbonate, dicalcium phosphate, calcium citrate, | 200-1000 | mg |
| calcium gluconate, calcium amino acid polysaccharide complex) | | |
| Iron (as iron amino acid polysaccharide complex) | 0-18 | mg |
| Phosphorus (as dicalcium phosphate) | 0-300 | mg |
| Iodine (as iodine amino acid polysaccharide complex) | 100-300 | μg |
| Magnesium (as magnesium oxide, magnesium lactate, magnesium | 100-400 | mg |
| citrate, magnesium amino acid polysaccharide complex) | | |
| Zinc (zinc amino acid polysaccharide complex) | 5-30 | mg |
| Selenium (as selenium amino acid polysaccharide complex) | 35-150 | μg |
| Copper (copper amino acid polysaccharide complex) | 1-5 | mg |
| Manganese (as manganese amino acid polysaccharide complex) | 1-5 | mg |
| Chromium (as chromium amino acid polysaccharide complex) | 60-360 | μg |
| Molybdenum (as molybdenum amino acid polysaccharide complex) | 50-150 | μg |
| Boron (as boron amino acid polysaccharide complex) | 0-300 | μg |

TABLE 2

| | Total Daily | |
|---|---|---|
| | Dose | |
| Vitamin A (as beta carotene) | 1000 | IU |
| Vitamin C (as ascorbic acid) | 100 | mg |
| Vitamin D (as cholecalciferol) | 200 | IU |
| Vitamin E (as d-alpha tocopheryl succinate) | 30 | IU |
| Vitamin K (as phytonadione) | 28 | μg |
| Thiamin (as thiamin HCl) | 15 | mg |
| Riboflavin | 17 | mg |
| Niacin (as niacinamide) | 75 | mg |
| Vitamin B6 (as pyridoxine HCl) | 10 | mg |
| Folate (as folic acid) | 800 | μg |
| Vitamin B12 (as cyanocobalamin) | 12 | μg |
| Biotin (as d-biotin) | 300 | μg |
| Pantothenic Acid (as calcium pantothenate) | 20 | mg |
| Calcium (as calcium carbonate, dicalcium phosphate, calcium | 250 | mg |
| citrate, calcium gluconate, calcium amino acid polyfructose | | |
| complex) | | |
| Iron (as iron amino acid polyfructose complex) | 9 | mg |
| Phosphorous (as dicalcium phosphate) | 65 | mg |
| Iodine (as iodine amino acid polyfructose complex) | 150 | μg |
| Magnesium (as magnesium oxide, magnesium lactate, magnesium | 200 | mg |
| citrate, magnesium amino acid polyfructose complex) | | |
| Zinc (zinc amino acid polyfructose complex) | 15 | mg |
| Selenium (as selenium amino acid polyfructose complex) | 105 | μg |
| Copper (copper amino acid polyfructose complex) | 3 | mg |
| Manganese (as manganese amino acid polyfructose complex) | 2.5 | mg |
| Chromium (as chromium amino acid polyfructose complex) | 120 | μg |
| Molybdenum (as molybdenum amino acid polyfructose complex) | 75 | μg |
| Boron (as boron amino acid polyfructose complex) | 150 | μg |

TABLE 3

| | Total Daily | |
|---|---|---|
| | Dose | |
| Vitamin A (as beta carotene) | 3000 | IU |
| Vitamin C (as ascorbic acid) | 150 | mg |
| Vitamin D (as cholecalciferol) | 200 | IU |
| Vitamin E (as d-alpha tocopheryl succinate) | 30 | IU |
| Thiamin (as thiamin HCl) | 15 | mg |
| Riboflavin | 17 | mg |
| Niacin (as niacinamide) | 75 | mg |
| Vitamin B6 (as pyridoxine HCl) | 10 | mg |
| Folate (as folic acid) | 600 | μg |
| Vitamin B12 (as cyanocobalamin) | 12 | μg |
| Biotin (as d-biotin) | 60 | μg |
| Pantothenic Acid (as calcium pantothenate) | 20 | mg |
| Calcium (as calcium carbonate, dicalcium phosphate, | 250 | mg |
| calcium citrate, calcium gluconate, calcium amino | | |
| acid polyfructose complex) | | |
| Phosphorous (as dicalcium phosphate) | 65 | mg |
| Iodine (as iodine amino acid polyfructose complex) | 150 | μg |
| Magnesium (as magnesium oxide, magnesium lactate, | 200 | mg |
| magnesium citrate, magnesium amino acid polyfructose | | |
| complex) | | |
| Zinc (zinc amino acid polyfructose complex) | 15 | mg |
| Selenium (as selenium amino acid polyfructose | 105 | μg |
| complex) | | |
| Copper (copper amino acid polyfructose complex) | 3 | mg |
| Manganese (as manganese amino acid polyfructose | 4 | mg |
| complex) | | |
| Chromium (as chromium amino acid polyfructose | 120 | μg |
| complex) | | |
| Molybdenum (as molybdenum amino acid polyfructose | 75 | μg |
| complex) | | |
| Boron (as boron amino acid polyfructose complex) | 150 | μg |

TABLE 4

| | Total Daily | |
|---|---|---|
| | Dose | |
| Vitamin A (as beta carotene) | 2000 | IU |
| Vitamin C (as ascorbic acid) | 150 | mg |
| Vitamin D (as cholecalciferol) | 200 | IU |
| Vitamin E (as d-alpha tocopheryl succinate) | 30 | IU |
| Thiamin (as thiamin HCl) | 8.5 | mg |
| Riboflavin | 10 | mg |
| Niacin (as niacinamide) | 75 | mg |
| Vitamin B6 (as pyridoxine HCl) | 10 | mg |
| Folate (as folic acid) | 1000 | μg |
| Vitamin B12 (as cyanocobalamin) | 16 | μg |
| Biotin (as d-biotin) | 300 | μg |
| Pantothenic Acid (as calcium pantothenate) | 20 | mg |
| Calcium (as calcium carbonate, dicalcium phosphate, | 300 | mg |
| calcium citrate, calcium gluconate, calcium amino acid | | |
| polyfructose complex) | | |
| Iron (as iron amino acid polyfructose complex) | 9 | mg |
| Phosphorous (as dicalcium phosphate) | 65 | mg |
| Iodine (as iodine amino acid polyfructose complex) | 150 | μg |
| Magnesium (as magnesium oxide, magnesium lactate, | 225 | mg |
| magnesium citrate, magnesium amino acid polyfructose | | |
| complex) | | |
| Zinc (zinc amino acid polyfructose complex) | 15 | mg |
| Selenium (as selenium amino acid polyfructose | 105 | μg |
| complex) | | |
| Copper (copper amino acid polyfructose complex) | 3 | mg |
| Manganese (as manganese amino acid polyfructose | 2.5 | mg |
| complex) | | |
| Chromium (as chromium amino acid polyfructose | 120 | μg |
| complex) | | |
| Molybdenum (as molybdenum amino acid polyfructose | 75 | μg |
| complex) | | |
| Boron (as boron amino acid polyfructose complex) | 150 | μg |

TABLE 5

| | Total Daily | |
|---|---|---|
| | Dose | |
| Vitamin A (as beta carotene) | 2500 | IU |
| Vitamin C (as ascorbic acid) | 80 | mg |
| Vitamin D (as cholecalciferol) | 200 | IU |
| Vitamin E (as d-alpha tocopheryl succinate) | 15 | IU |
| Thiamin (as thiamin HCl) | 0.7 | mg |
| Riboflavin | 0.8 | mg |
| Niacin (as niacinamide) | 9 | mg |
| Vitamin B6 (as pyridoxine HCl) | 1.05 | mg |
| Folate (as folic acid) | 200 | μg |
| Vitamin B12 (as cyanocobalamin) | 3 | μg |
| Biotin (as d-biotin) | 30 | μg |
| Pantothenic Acid (as calcium pantothenate) | 5 | mg |
| Calcium (as calcium carbonate, dicalcium phosphate, | 160 | mg |
| calcium citrate, calcium gluconate, calcium amino | | |
| acid polyfructose complex) | | |
| Iron (as iron amino acid polyfructose complex) | 5 | mg |
| Phosphorous (as dicalcium phosphate) | 20 | mg |
| Iodine (as iodine amino acid polyfructose complex) | 70 | μg |
| Magnesium (as magnesium oxide, magnesium lactate, | 40 | mg |
| magnesium citrate, magnesium amino acid polyfructose | | |
| complex) | | |
| Zinc (zinc amino acid polyfructose complex) | 4 | mg |
| Copper (copper amino acid polyfructose complex) | 0.5 | mg |
| Manganese (as manganese amino acid polyfructose | 0.5 | mg |
| complex) | | |
| Chromium (as chromium amino acid polyfructose | 10 | μg |
| complex) | | |
| Molybdenum (as molybdenum amino acid polyfructose | 10 | μg |
| complex) | | |

TABLE 6

| | Total Daily | |
|---|---|---|
| | Dose | |
| Vitamin A (as beta carotene) | 5000 | IU |
| Vitamin C (as ascorbic acid) | 160 | mg |
| Vitamin D (as cholecalciferol) | 400 | IU |
| Vitamin E (as d-alpha tocopheryl succinate) | 30 | IU |
| Thiamin (as thiamin HCl) | 1.4 | mg |
| Riboflavin | 1.6 | mg |
| Niacin (as niacinamide) | 18 | mg |
| Vitamin B6 (as pyridoxine HCl) | 2.1 | mg |
| Folate (as folic acid) | 400 | μg |
| Vitamin B12 (as cyanocobalamin) | 6 | μg |
| Biotin (as d-biotin) | 60 | μg |
| Pantothenic Acid (as calcium pantothenate) | 10 | mg |
| Calcium (as calcium carbonate, dicalcium phosphate, | 320 | mg |
| calcium citrate, calcium gluconate, calcium amino | | |
| acid polyfructose complex) | | |
| Iron (as iron amino acid polyfructose complex) | 10 | mg |
| Phosphorous (as dicalcium phosphate) | 40 | mg |
| Iodine (as iodine amino acid polyfructose complex) | 140 | μg |
| Magnesium (as magnesium oxide, magnesium lactate, | 80 | mg |
| magnesium citrate, magnesium amino acid polyfructose | | |
| complex) | | |
| Zinc (zinc amino acid polyfructose complex) | 8 | mg |
| Copper (copper amino acid polyfructose complex) | 1 | mg |
| Manganese (as manganese amino acid polyfructose | 1 | mg |
| complex) | | |
| Chromium (as chromium amino acid polyfructose | 20 | μg |
| complex) | | |
| Molybdenum (as molybdenum amino acid polyfructose | 20 | μg |
| complex) | | |

TABLE 7

| | Total Daily | |
|---|---|---|
| | Dose | |
| Vitamin A (as beta carotene) | 500 | IU |
| Vitamin C (as ascorbic acid) | 150 | mg |
| Vitamin D (as cholecalciferol) | 200 | IU |
| Vitamin E (as d-alpha tocopheryl succinate) | 45 | IU |
| Thiamin (as thiamin HCl) | 7.5 | mg |
| Riboflavin | 8.5 | mg |
| Niacin (as niacinamide) | 75 | mg |
| Vitamin B6 (as pyridoxine HCl) | 10 | mg |
| Folate (as folic acid) | 600 | μg |
| Vitamin B12 (as cyanocobalamin) | 12 | μg |
| Biotin (as d-biotin) | 60 | μg |
| Pantothenic Acid (as calcium pantothenate) | 15 | mg |
| Calcium (as calcium carbonate, dicalcium phosphate, | 250 | mg |
| calcium citrate, calcium gluconate, calcium amino acid | | |
| polyfructose complex) | | |
| Phosphorous (as dicalcium phosphate) | 65 | mg |
| Iodine (as iodine amino acid polyfructose complex) | 150 | μg |
| Magnesium (as magnesium oxide, magnesium lactate, | 200 | mg |
| magnesium citrate, magnesium amino acid polyfructose | | |
| complex) | | |
| Zinc (zinc amino acid polyfructose complex) | 15 | mg |
| Selenium (as selenium amino acid polyfructose | 105 | μg |
| complex) | | |
| Copper (copper amino acid polyfructose complex) | 3 | mg |
| Manganese (as manganese amino acid polyfructose | 3 | mg |
| complex) | | |
| Chromium (as chromium amino acid polyfructose | 180 | μg |
| complex) | | |
| Molybdenum (as molybdenum amino acid polyfructose | 90 | μg |
| complex) | | |
| Boron (as boron amino acid polyfructose complex) | 150 | μg |

TABLE 8

| | Total Daily | |
|---|---|---|
| | Dose | |
| Vitamin D (as cholecalciferol) | 300 | IU |
| Calcium (as calcium carbonate, dicalcium phosphate, | 750 | mg |
| calcium citrate, calcium gluconate, calcium amino | | |
| acid polyfructose complex) | | |
| Phosphorous (as dicalcium phosphate) | 100 | mg |
| Magnesium (as magnesium oxide, magnesium lactate, | 150 | mg |
| magnesium citrate, magnesium amino acid polyfructose | | |
| complex) | | |

TABLE 9

| | Total Daily | |
|---|---|---|
| | Dose | |
| Vitamin D (as cholecalciferol) | 400 | IU |
| Calcium (as calcium carbonate, dicalcium phosphate, | 1000 | mg |
| calcium citrate, calcium gluconate, calcium amino | | |
| acid polyfructose complex) | | |
| Phosphorous (as dicalcium phosphate) | 130 | mg |
| Magnesium (as magnesium oxide, magnesium lactate, | 200 | mg |
| magnesium citrate, magnesium amino acid polyfructose | | |
| complex) | | |

TABLE 10

| | Total Daily | |
|---|---|---|
| | Dose | |
| Vitamin D (as cholecalciferol) | 200 | IU |
| Calcium (as calcium carbonate, dicalcium phosphate, | 500 | mg |
| calcium citrate, calcium gluconate, calcium amino | | |
| acid polyfructose complex) | | |
| Phosphorous (as dicalcium phosphate) | 64 | mg |
| Magnesium (as magnesium oxide, magnesium lactate, | 100 | mg |
| magnesium citrate, magnesium amino acid polyfructose | | |
| complex) | | |

TABLE 11

| | Total Daily | |
|---|---|---|
| | Dose | |
| Vitamin D (as cholecalciferol) | 500 | IU |
| Calcium (as calcium carbonate, dicalcium phosphate, | 1250 | mg |
| calcium citrate, calcium gluconate, calcium amino | | |
| acid polyfructose complex) | | |
| Phosphorous (as dicalcium phosphate) | 160 | mg |
| Magnesium (as magnesium oxide, magnesium lactate, | 250 | mg |
| magnesium citrate, magnesium amino acid polyfructose | | |
| complex) | | |

TABLE 12

| | | |
|---|---|---|
| Calcium Carbonate | 200-300 | mg |
| Calcium Citrate | 5-10 | mg |
| Calcium Gluconate | 10-15 | mg |
| Calcium Lactate | 10-20 | mg |
| Magnesium Carbonate | 150-250 | mg |
| Magnesium Citrate | 50-100 | mg |
| Magnesium Lactate | 50-100 | mg |
| Chromium Chloride | 0.1-0.5 | mg |
| Copper Sulfate | 1-5 | mg |
| Manganese Sulfate | 2-5 | mg |
| Sodium Selenite | 0.01-0.05 | mg |
| Zinc Sulfate | 10-15 | mg |
| Ferrous Sulfate | 15-20 | mg |

This document also provides methods for making the complexes and compositions described herein. In some cases, the methods provided herein can consist essentially of particular steps. By “consisting essentially of” with regard to a method is meant that the method includes one or more specified steps, and can contain additional steps that do not materially affect the basic and novel characteristics of the method.

In some cases, the mineral-amino acid/polysaccharide complexes described herein can be prepared by heating a mixture of water, one or more mineral-amino acid compounds, and one or more polysaccharides at a temperature from about 22° C. to about 82° C. (e.g., about 22° C., about 27° C., about 32° C., about 37° C., about 42° C., about 47° C., about 47° C., about 52° C., about 57° C., about 62° C., about 67° C., about 72° C., about 77° C., about 82° C., about 22° C. to about 27° C., about 27° C. to about 32° C., about 32° C. to about 37° C., about 37° C. to about 42° C., about 42° C. to about 47° C., about 47° C. to about 52° C., about 52° C. to about 57° C., or about 57° C. to about 62° C., about 62° C. to about 67° C., about 67° C. to about 72° C., about 72° C. to about 77° C., or about 77° C. to about 82° C.). The mixture of water, one or more mineral-amino acid compounds, and one or more polysaccharides can be heated for any suitable length of time. For example, a mixture of water, one or more mineral-amino acid compounds, and one or more polysaccharides can be heated from about 5 minutes to about 30 minutes (e.g., about 5 minutes to about 10 minutes, about 10 minutes to about 15 minutes, about 15 minutes to about 20 minutes, about 20 minutes to about 25 minutes, or about 25 minutes to about 30 minutes). In some cases, after the heating step, the resulting complexes can be dried to reduce the moisture content. For example, the complexes can be dried to a moisture content of less than about 15% (e.g., less than about 14%, less than about 12%, less than about 10%, less than about 8%, less than about 5%, or less than about 2%). Any suitable method can be used to dry the complexes. It is noted that the drying time and temperature can vary depending on the mineral in the complex being dried. For example, complexes containing zinc may be dried at a higher temperature and/or for a longer time than complexes containing other minerals.

In some cases, a combination of water, one or more mineral amino acid compounds, and one or more polysaccharides can be mixed (e.g., in a high shear mixer) at room temperature, and then heated in a drying oven to achieve a moisture content of less than about 15% (e.g., less than about 14%, less than about 12%, less than about 10%, less than about 8%, less than about 5%, or less than about 2%). The mixture can be heated for any suitable length of time to achieve a desired moisture content. For example, a mixture of water, one or more mineral-amino acid compounds, and one or more polysaccharides can be heated from about 30 minutes to about 8 hours (e.g., about 30 minutes to about 60 minutes, about 1 hour to about 2 hours, about 2 hours to about 4 hours, about 4 hours to about 6 hours, or about 6 hours to about 8 hours).

In some cases, the methods provided herein for making the presently described coated minerals or complexes can include one or more of the following steps.

Initially, one or more minerals such as zinc, manganese, iron, copper, selenium, chromium and the like can be independently chelated with an amino acid (e.g., glycine or aspartic acid) in the presence of sodium hydroxide via granulation with water at a temperature of about 65° C. to about 85° C. (e.g., about 65° C. to about 70° C., about 70° C. to about 75° C., about 75° C. to about 80° C., or about 80° C. to about 85° C.). Once the reaction process is complete, the mineral-amino acid compounds can be dried at a temperature between 40° C. and 80° C., and then can be milled to a particle size of about 10 to 200 mesh (e.g., 10 to 30 mesh, 30 to 50 mesh, 50 to 70 mesh, 70 to 80 mesh, 80 to 90 mesh, 90 to 100 mesh, 100 to 120 mesh, 120 to 140 mesh, 140 to 160 mesh, 160 to 180 mesh, or 180 to 200 mesh).

In some cases, the mineral-amino acid compounds generated as described above (or commercially available mineral-amino acid material that is fully or partially chelated and meets the 10 to 200 mesh particle size criteria) can be combined with a polysaccharide (e.g., polyfructose and/or inulin) and a coating material (e.g., stearic acid, sodium alginate, or any other suitable coating material).

In some cases, the combined mineral-amino acid compounds and (when included) the polysaccharide, with or without the coating material, can be mixed using a method (e.g., dry blending) that yields a homogeneous mixture of the two ingredients. It is to be noted that in some cases the coating material can be included in the blend, while in other cases the coating material can be dissolved in a granulation solvent and introduced after this step. In general, the more vigorously the ingredients are agitated, the better they will bond together.

The blended materials then can be granulated (e.g., in a high shear granulator), which can provide additional high intensity blending of the components of the complexes. In some cases, the materials can be blended (e.g., dry blended) and granulated in a fluid bed, using either a top spray or a bottom spray configuration. In such cases, the coating material can be added during the blending stage (e.g., during a dry blending stage), added to the granulation solution, or both.

The granulated material can then be subjected to wet milling in order to reduce the particle size of the material to 10 mesh or smaller (e.g., 10 to 20 mesh, 20 to 30 mesh, 30 to 40 mesh, 40 to 50 mesh, 50 to 60 mesh, 60 to 70 mesh, 70 to 80 mesh, 80 to 90 mesh, 90 to 100 mesh, or smaller than 100 mesh). The wet milling can be carried out with, for example, a conical mill.

The milled material then can be dried. In some cases, for example, the milled material can be dried in a fluid bed dryer at a temperature from about 20° C. to about 100° C. (e.g., about 20° C. to about 40° C., about 40° C. to about 60° C., about 60° C. to about 80° C., or about 80° C. to about 100° C.) for about 30 minutes to about 8 hours (e.g., about 30 minutes to about 60 minutes, about 1 hour to about 2 hours, about 2 hours to about 4 hours, about 4 hours to about 6 hours, or about 6 hours to about 8 hours), which can achieve drying of the material in a relatively gentle manner, and can avoid the need for a further milling step at the end of the process, thus preserving the integrity of the coated particles. In some cases, the dried particles can be screened through a vibrating or centrifugal sifter utilizing a screen size between 10 and 200 mesh (e.g., 10 to 30 mesh, 30 to 50 mesh, 50 to 70 mesh, 70 to 80 mesh, 80 to 90 mesh, 90 to 100 mesh, or 100 to 120 mesh, 120 to 140 mesh, 140 to 160 mesh, 160 to 180 mesh, or 180 to 200 mesh).

When non-chelated minerals or mineral salts are used, the minerals or mineral salts optionally can be milled to a particle size of about 10 to 200 mesh (e.g., 10 to 30 mesh, 30 to 50 mesh, 50 to 70 mesh, 70 to 80 mesh, 80 to 90 mesh, 90 to 100 mesh, 100 to 120 mesh, 120 to 140 mesh, 140 to 160 mesh, 160 to 180 mesh, or 180 to 200 mesh). Particles of one or more minerals or mineral salts (e.g., one or more of zinc, copper, selenium, manganese, chromium, iron, and salts thereof can be combined with a coating material (e.g., stearic acid, sodium alginate, or any other suitable coating material). In some cases, minerals or mineral salts, with or without a coating material, can be mixed using a method (e.g., dry blending) that yields a homogeneous mixture. It is to be noted that in some cases the coating material can be included in the blend, while in other cases the coating material can be dissolved in a granulation solvent and introduced after this step. In general, the more vigorously the ingredients are agitated, the better they will bond together.

The blended mineral or mineral salt materials then can be granulated (e.g., in a high shear granulator), which can provide additional high intensity blending of the components of the complexes. In some cases, the materials can be blended (e.g., dry blended) and granulated in a fluid bed, using either a top spray or a bottom spray configuration. In such cases, the coating material can be added during the blending stage (e.g., during a dry blending stage), added to the granulation solution, or both.

The granulated material can then be subjected to wet milling in order to reduce the particle size of the material to 10 mesh or smaller (e.g., 10 to 20 mesh, 20 to 30 mesh, 30 to 40 mesh, 40 to 50 mesh, 50 to 60 mesh, 60 to 70 mesh, 70 to 80 mesh, 80 to 90 mesh, 90 to 100 mesh, or smaller than 100 mesh). The wet milling can be carried out with, for example, a conical mill.

The milled material then can be dried. In some cases, for example, the milled material can be dried in a fluid bed dryer at a temperature from about 20° C. to about 100° C. (e.g., about 20° C. to about 40° C., about 40° C. to about 60° C., about 60° C. to about 80° C., or about 80° C. to about 100° C.) for about 30 minutes to about 8 hours (e.g., about 30 minutes to about 60 minutes, about 1 hour to about 2 hours, about 2 hours to about 4 hours, about 4 hours to about 6 hours, or about 6 hours to about 8 hours), which can achieve drying of the material in a relatively gentle manner, and can avoid the need for a further milling step at the end of the process, thus preserving the integrity of the coated particles. In some cases, the dried particles can be screened through a vibrating or centrifugal sifter utilizing a screen size between 10 and 200 mesh (e.g., 10 to 30 mesh, 30 to 50 mesh, 50 to 70 mesh, 70 to 80 mesh, 80 to 90 mesh, 90 to 100 mesh, or 100 to 120 mesh, 120 to 140 mesh, 140 to 160 mesh, 160 to 180 mesh, or 180 to 200 mesh).

Mineral-amino acid compounds and mineral-amino acid/polysaccharide complexes described herein can reduce the formation of free radicals in the intestinal tract, as compared to uncomplexed minerals (e.g., a mineral sulfate, chloride, citrate, or gluconate). In some cases, a mineral-amino acid/polysaccharide complex can be an iron-amino acid/polyfructose complex or a copper-amino acid/polyfructose complex, or an iron-glycinate/inulin, iron-aspartic acid/inulin, or copper-glycinate/inulin complex. In some cases, the uncomplexed mineral can be an inorganic salt (e.g., a mineral sulfate, chloride or oxide). A reduction in the formation of free radicals can include, for example, a reduction in the formation of reactive oxygen species (ROS) such as hydroxyl radicals, peroxide, and superoxide radicals. ROS can initiate radical chain reactions within the body. For example, an uncomplexed mineral provided in a dietary supplement can catalyze the formation of ROS that can oxidize one or more antioxidants from the dietary supplement (e.g., ascorbic acid, tocopherols, carotenoids, lipoic acid, natural plant phenols and flavonoids, and polyunsaturated fatty acids). Without being bound by theory, a complexed mineral may be protected by the amino acid and/or the polysaccharide, and thus is less able to catalyze free radical formation. When complexed minerals are administered in the form of a dietary supplement, a reduction in the formation of free radicals can decrease the oxidation of any antioxidants that also are present in the dietary supplement.

Accordingly, this document further provides methods for providing a mammal with a mineral and antioxidant mixture having reduced mineral-catalyzed oxidation. In some cases, the methods can include administering to a mammal a composition containing a mineral and an antioxidant, where the mineral is in the form of a mineral-amino acid complex or a mineral-amino acid/polysaccharide complex as described herein, and where the mineral-amino acid complex or the mineral-amino acid polysaccharide complex exhibits reduced mineral-catalyzed oxidation of the antioxidant as compared to a corresponding uncomplexed mineral or a mineral administered as an inorganic salt. The reduced oxidation can be, for example, at least a 10% decrease (e.g., at least 20%, at least 25%, at least 30%, at least 40%, at least 50%, at least 60%, at least 70%, at least 80%, or at least 90% decrease) in oxidation as compared to the level of oxidation that would occur with a corresponding uncomplexed mineral or a mineral administered as an inorganic salt.

In addition to stabilization of antioxidants present in a dietary supplement, a reduction in the formation of free radicals also can reduce the oxidation of bio-molecules such as lipids, proteins, and DNA. In some cases, oxidative damage of bio-molecules can lead to formation of many different intermediate radicals, including toxic and/or pathogenic products. Such radical species can change the structure and/or function of bio-molecules, and can provoke degenerative diseases, inflammation, cancer, and arteriosclerosis, for example.

This disclosure provides methods for reducing the formation of free radicals, reducing the amount of free radicals, and reducing antioxidant oxidation in a mammal or a cell using one or more minerals or mineral salts, mineral-amino acid complexes, or mineral-amino acid/polysaccharide complexes as described herein. For example, complexes such as iron-amino acid/polysaccharide or copper-amino acid/polysaccharide complexes can be used to reduce the formation and/or amount of free radicals, and to reduce antioxidant oxidation in a mammal or a cell (e.g., an intestinal cell). In some cases, the reduced formation and/or amount of free radicals and the decreased antioxidant oxidation can occur in a mammal's intestine after ingestion of a composition provided herein that contains one or more mineral-amino acid/polysaccharide complexes.

In some cases, the methods provided herein can include contacting a cell in vitro with a composition containing minerals or mineral salts, mineral-amino acid complexes, or mineral-amino acid/polysaccharide complexes as described herein, where the contacting results in reduced free radical formation, a reduced amount of a free radical, and/or decreased oxidation of an antioxidant (e.g., an antioxidant selected from ascorbic acid, tocopherols, carotenoids, lipoic acid, natural plant phenols and flavonoids, and polyunsaturated fatty acids) in the cell. For example, the contacting can be performed using cells within which a free radical or an antioxidant is present. In some cases, an in vitro method can be performed in a cell free medium. Uses of such in vitro methods for reducing formation of a free radical include, without limitation, use in a screening assay (for example, where the mineral-amino acid/polysaccharide complex is used as a positive control or standard for comparison to compounds of unknown activity or potency in reducing free radical formation).

In some cases, the methods can be used in vivo, for example, to reduce free radical formation, reduce the amount of a free radical, and/or decrease antioxidant oxidation. Such methods can be performed by, for example, contacting a cell in vivo (e.g., in a mammal) with two or more minerals or mineral salts, mineral-amino acid complexes, or mineral-amino acid/polysaccharide complexes as described herein, where at least one of the minerals or mineral salts, mineral-amino acid complexes, or mineral-amino acid/polysaccharide complexes has a slow release coating. In some cases, the methods can include administering to a mammal a composition containing two or more mineral-amino acid/polysaccharide complexes, where at least one of the mineral-amino acid/polysaccharide complexes has a slow release coating, and where the composition is administered in an effective amount to, for example, reduce free radical formation. The complexes administered to the cell or the mammal also can include a mineral-amino acid/polysaccharide complex that does not have a slow release coating.

In some cases, the methods provided herein can be used to treat or reduce the likelihood of a disease or clinical condition (e.g., a degenerative disease, inflammation, cancer, or arteriosclerosis) in a mammal by reducing free radical formation and/or free radical amounts in the mammal. The methods can include administering to a mammal an effective amount of a composition provided herein, where the composition includes two or more minerals or mineral salts, mineral-amino acid complexes, or mineral-amino acid/polysaccharide complexes, and where at least one of the minerals or mineral salts, mineral-amino acid complexes, or mineral-amino acid/polysaccharide complexes has a slow release coating and at least one of the mineral-amino acid complexes or mineral-amino acid/polysaccharide complexes does not have a slow release coating. An effective amount of a composition or a complex can be, for example, a daily dose as set forth in the tables herein.

### Exemplary Embodiments

Embodiment 1 is a composition comprising a first complex that comprises a first compound conjugated to a first polysaccharide, wherein the first compound comprises a first mineral and a first amino acid, and wherein the first complex is coated with a slow release coating.

Embodiment 2 is the composition of embodiment 1, wherein the first mineral is selected from the group consisting of zinc, copper, selenium, manganese, chromium, and iron.

Embodiment 3 is the composition of embodiment 1 or embodiment 2, wherein the first amino acid is selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine.

Embodiment 4 is the composition of embodiment 1 or embodiment 2, wherein the first amino acid is selected from the group consisting of L-glycine and L-aspartic acid.

Embodiment 5 is the composition of any one of embodiments 1 to 4, wherein the first polysaccharide is selected from the group consisting of cellulose, polyhexoses, polypentoses, polydextrose, starch, polygalactan, polymannan, chitin, chitosan, chondroitin, polyfructose, inulin, pectin, and derivatives thereof.

Embodiment 6 is the composition of any one of embodiments 1 to 4, wherein the first polysaccharide is selected from the group consisting of a cellulose derivative, polyfructose, inulin, and polydextrose.

Embodiment 7 is the composition of any one of embodiments 1 to 6, wherein the slow release coating comprises sodium alginate, stearic acid, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, monoglycerides, diglycerides, or any combination thereof.

Embodiment 8 is the composition of any one of embodiments 1 to 7, wherein the first complex is selected from the group consisting of: a zinc-amino acid/polyfructose complex, a copper-amino acid/polyfructose complex, a selenium-amino acid/polyfructose complex, a manganese-amino acid/polyfructose complex, a chromium-amino acid/polyfructose complex, and an iron-amino acid/polyfructose complex.

Embodiment 9 is the composition of any one of embodiments 1 to 8, further comprising one or more of vitamin A, vitamin C, vitamin D, vitamin E, vitamin K, thiamin, riboflavin, niacin, vitamin B6, folate, vitamin B12, biotin, pantothenic acid, and phosphorous.

Embodiment 10 is the composition of any one of embodiments 1 to 9, further comprising a second complex that comprises a second compound conjugated to a second polysaccharide, wherein the second compound comprises a second mineral and a second amino acid, wherein the second mineral is different from the first mineral, and wherein the second complex is not coated with a slow release coating.

Embodiment 11 is the composition of embodiment 10, wherein the second mineral is selected from the group consisting of magnesium, calcium, boron, iodine, potassium, and molybdenum.

Embodiment 12 is the composition of embodiment 10 or embodiment 11, wherein the first amino acid and the second amino acid are selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine.

Embodiment 13 is the composition of embodiment 10 or embodiment 11, wherein the first amino acid and the second amino acid are selected from the group consisting of L-glycine and L-aspartic acid.

Embodiment 14 is the composition of any one of embodiments 10 to 13, wherein the first amino acid and the second amino acid are the same.

Embodiment 15 is the composition of any one of embodiments 10 to 13, wherein the first amino acid and the second amino acid are different.

Embodiment 16 is the composition of any one of embodiments 10 to 15, wherein the first polysaccharide and the second polysaccharide are selected from the group consisting of cellulose, polyhexoses, polypentoses, polydextrose, starch, polygalactan, polymannan, chitin, chitosan, chondroitin, polyfructose, inulin, pectin, and derivatives thereof.

Embodiment 17 is the composition of any one of embodiments 10 to 16, wherein the first polysaccharide and the second polysaccharide are the same.

Embodiment 18 is the composition of any one of embodiments 10 to 16, wherein the first polysaccharide and the second polysaccharide are different.

Embodiment 19 is the composition of any one of embodiments 10 to 18, wherein the first polysaccharide and the second polysaccharide are selected from the group consisting of a cellulose derivative, polyfructose, inulin, and polydextrose.

Embodiment 20 is the composition of any one of embodiments 10 to 19, wherein the first complex is selected from the group consisting of: a zinc-amino acid/polyfructose complex, a copper-amino acid/polyfructose complex, a selenium-amino acid/polyfructose complex, a manganese-amino acid/polyfructose complex, a chromium-amino acid/polyfructose complex, and an iron-amino acid/polyfructose complex, and wherein the second complex is selected from the group consisting of: a calcium-amino acid/polyfructose complex, an iodine-amino acid/polyfructose complex, a magnesium-amino acid/polyfructose complex, a potassium-amino acid/polyfructose complex, a molybdenum-amino acid/polyfructose complex, and a boron-amino acid/polyfructose complex.

Embodiment 21 is the composition of any one of embodiments 10 to 20, further comprising one or more of vitamin A, vitamin C, vitamin D, vitamin E, vitamin K, thiamin, riboflavin, niacin, vitamin B6, folate, vitamin B12, biotin, pantothenic acid, and phosphorous.

Embodiment 22 is a method for making a coated mineral-amino acid/polysaccharide complex, wherein the method comprises dry-blending a combination of (a) one or more mineral-amino acid compounds having a particle size smaller than about 80 mesh with (b) a polysaccharide and (c) a slow release coating material; granulating the dry-blended combination to generate a granulated material; and drying the granulated material.

Embodiment 23 is the method of embodiment 22, wherein the drying comprises using a fluid bed dryer.

Embodiment 24 is the method of embodiment 22 or embodiment 23, further comprising wet milling the granulated material to reduce the particle size of the material to 10 mesh or smaller.

Embodiment 25 is a composition comprising a first compound that comprises a first mineral coupled to a first amino acid, wherein the first compound is coated with a slow release coating.

Embodiment 26 is the composition of embodiment 25, wherein the first mineral is selected from the group consisting of zinc, copper, selenium, manganese, chromium, and iron.

Embodiment 27 is the composition of embodiment 25 or embodiment 26, wherein the first amino acid is selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine.

Embodiment 28 is the composition of embodiment 25 or embodiment 26, wherein the first amino acid is selected from the group consisting of L-glycine and L-aspartic acid.

Embodiment 29 is the composition of any one of embodiments 25 to 28, wherein the slow release coating comprises sodium alginate, stearic acid, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, monoglycerides, diglycerides, or any combination thereof.

Embodiment 30 is the composition of any one of embodiments 25 to 29, wherein the first compound is selected from the group consisting of: a zinc-amino acid, a copper-amino acid, a selenium-amino acid, a manganese-amino acid, a chromium-amino acid, and an iron-amino acid.

Embodiment 31 is the composition of any one of embodiments 25 to 30, further comprising one or more of vitamin A, vitamin C, vitamin D, vitamin E, vitamin K, thiamin, riboflavin, niacin, vitamin B6, folate, vitamin B12, biotin, pantothenic acid, and phosphorous.

Embodiment 32 is the composition of any one of embodiment 25 to 31, further comprising a second compound that comprises a second mineral coupled to a second amino acid, wherein the second mineral is different from the first mineral, and wherein the second compound is not coated with a slow release coating.

Embodiment 33 is the composition of embodiment 32, wherein the second mineral is selected from the group consisting of magnesium, calcium, boron, iodine, potassium, and molybdenum.

Embodiment 34 is the composition of embodiment 32 or embodiment 33, wherein the first amino acid and the second amino acid are selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine.

Embodiment 35 is the composition of embodiment 32 or embodiment 33, wherein the first amino acid and the second amino acid are selected from the group consisting of L-glycine and L-aspartic acid.

Embodiment 36 is the composition of any one of embodiments 32 to 35, wherein the first amino acid and the second amino acid are the same.

Embodiment 37 is the composition of any one of embodiments 32 to 35, wherein the first amino acid and the second amino acid are different.

Embodiment 38 is the composition of any one of embodiments 32 to 37, wherein the first compound is selected from the group consisting of: a zinc-amino acid, a copper-amino acid, a selenium-amino acid, a manganese-amino acid, a chromium-amino acid, and an iron-amino acid, and wherein the second compound is selected from the group consisting of: a calcium-amino acid, an iodine-amino acid, a magnesium-amino acid, a potassium-amino acid, a molybdenum-amino acid, and a boron-amino acid.

Embodiment 39 is the composition of any one of embodiments 32 to 38, further comprising one or more of vitamin A, vitamin C, vitamin D, vitamin E, vitamin K, thiamin, riboflavin, niacin, vitamin B6, folate, vitamin B12, biotin, pantothenic acid, and phosphorous.

Embodiment 40 is a method for making a coated mineral-amino acid compound, wherein the method comprises: dry-blending a combination of (a) one or more mineral-amino acid compounds having a particle size smaller than about 80 mesh with (b) a slow release coating material; granulating the dry-blended combination to generate a granulated material; and drying the granulated material.

Embodiment 41 is the method of embodiment 40, wherein the drying comprises using a fluid bed dryer.

Embodiment 42 is the method of embodiment 40 or embodiment 41, further comprising wet milling the granulated material to reduce the particle size of the material to 10 mesh or smaller.

Embodiment 43 is a method for making a coated mineral-amino acid compound, wherein the method comprises: dry-blending one or more mineral-amino acid compounds having a particle size smaller than about 80 mesh in a fluid bed; granulating the dry-blended compounds in the fluid bed; and adding a slow release coating material to the mineral-amino acid compounds during the dry-blending step, during the granulating step, or during the dry-blending step and the granulating step.

Embodiment 44 is the method of embodiment 43, further comprising drying the granulated material.

Embodiment 45 is the method of embodiment 43 or embodiment 44, wherein the fluid bed is in a top spray configuration.

Embodiment 46 is the method of embodiment 43 or embodiment 44, wherein the fluid bed is in a bottom spray configuration.

Embodiment 47 is a composition comprising particles of one or more minerals or mineral salts, wherein the particles are coated with a slow release coating.

Embodiment 48 is the composition of embodiment 47, wherein the one or more minerals or mineral salts comprise one or more of zinc, copper, selenium, manganese, chromium, iron, a zinc salt, a copper salt, a selenium salt, a manganese salt, a chromium salt, and an iron salt.

Embodiment 49 is the composition of embodiment 47 or embodiment 48, further comprising particles of one or more minerals or mineral salts that are not coated with a slow release coating.

Embodiment 50 is the composition of embodiment 49, wherein the particles that are not coated comprise one or more of magnesium, calcium, boron, iodine, potassium, molybdenum, sodium, a magnesium salt, a calcium salt, a boron salt, an iodine salt, a potassium salt, a molybdenum salt, and a sodium salt.

Embodiment 51 is the composition of embodiment 49 or embodiment 50, wherein the composition comprises: a chromium salt, a copper salt, a manganese salt, a zinc salt, an iron salt, a sodium salt, a magnesium salt, and a calcium salt.

Embodiment 52 is the composition of any one of embodiments 47 to 51, wherein the slow release coating comprises sodium alginate, stearic acid, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, monoglycerides, diglycerides, or any combination thereof.

The invention will be further described in the following example, which does not limit the scope of the invention described in the claims.

### Example

Coated magnesium-amino acid/polysaccharide samples were prepared in a top spray fluid bed using sodium alginate as a film-forming polymer. The method included a high shear granulation step followed by a fluid bed coating step.

The high shear granulation step was done in a Kitchen Aid mixer, in which 500 grams of magnesium bisglycinate was granulated utilizing a 2% solution of 40° C. water and sodium alginate. The amount of granulating solution used was 125 grams, yielding an input of sodium alginate into the granulation of 2.5 grams, or 0.5% by weight. Granulation was followed by drying at 65° C. for 12 hours. After drying, the material was passed through a 20 mesh screen to remove any lumps.

For the coating step, the material was then transferred to a Freund-Vector fluid bed (model #FL-Multi-1; Freund-Vector Corp., Marion, IA), and spray coated utilizing the following processing parameters:

| Spray coating solution: | 750 g water and 10 g sodium alginate | |
|---|---|---|
| Inlet air temperature: | 70° | C. |
| Product Temperature: | 45° | C. |
| Airflow setting: | 1.25 | CFU/m |
| Spray pump RPM | 6.3 | |
| Nozzle air pressure | 9.5 | psi |

Spraying was conducted for 9.2 hours, at which time the spraying was stopped and drying was continued at the 70° C. inlet temperature for 30 minutes. The material was allowed to cool for 45 minutes, and was then passed through a 12 mesh screen to remove lumps. The total sodium alginate content in the finished product was 2.5% by weight.

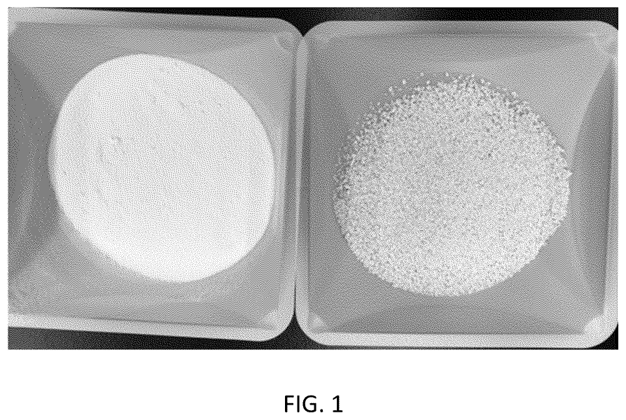

Images of the coated vs. uncoated material are shown in FIG. 1. When 25 g of coated or uncoated material was mixed with 450 mL of 37° C. water, the uncoated material was substantially dissolved within about 20 minutes, while the coated material was mostly undissolved in the same time period. Thus, the granulation and coating delayed the release of the highly soluble magnesium chelate into solution.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising a first complex that comprises a first compound conjugated to a first polysaccharide, wherein the first compound comprises a first mineral and a first amino acid, and wherein the first complex is coated with a slow release coating.

2. The composition of claim 1, wherein the first mineral is selected from the group consisting of zinc, copper, selenium, manganese, chromium, and iron.

3. The composition of claim 1, wherein the first amino acid is selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine.

4. The composition of claim 1, wherein the first polysaccharide is selected from the group consisting of cellulose, polyhexoses, polypentoses, polydextrose, starch, polygalactan, polymannan, chitin, chitosan, chondroitin, polyfructose, inulin, pectin, and derivatives thereof.

5. The composition of claim 1, wherein the slow release coating comprises sodium alginate, stearic acid, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, monoglycerides, diglycerides, or any combination thereof.

6. The composition of claim 1,
wherein the first complex is selected from the group consisting of:
a zinc-amino acid/polyfructose complex,
a copper-amino acid/polyfructose complex,
a selenium-amino acid/polyfructose complex,
a manganese-amino acid/polyfructose complex,
a chromium-amino acid/polyfructose complex, and
an iron-amino acid/polyfructose complex.

7. The composition of claim 1, further comprising one or more of vitamin A, vitamin C, vitamin D, vitamin E, vitamin K, thiamin, riboflavin, niacin, vitamin B6, folate, vitamin B12, biotin, pantothenic acid, and phosphorous.

8. The composition of claim 1, further comprising a second complex that comprises a second compound conjugated to a second polysaccharide, wherein the second compound comprises a second mineral and a second amino acid, wherein the second mineral is different from the first mineral, and wherein the second complex is not coated with a slow release coating.

9. The composition of claim 8,
wherein the first complex is selected from the group consisting of:
a zinc-amino acid/polyfructose complex,
a copper-amino acid/polyfructose complex,
a selenium-amino acid/polyfructose complex,
a manganese-amino acid/polyfructose complex,
a chromium-amino acid/polyfructose complex, and
an iron-amino acid/polyfructose complex,
and wherein the second complex is selected from the group consisting of:
a calcium-amino acid/polyfructose complex,
an iodine-amino acid/polyfructose complex,
a magnesium-amino acid/polyfructose complex,
a potassium-amino acid/polyfructose complex,
a molybdenum-amino acid/polyfructose complex, and
a boron-amino acid/polyfructose complex.

10. The composition of claim 8, further comprising one or more of vitamin A, vitamin C, vitamin D, vitamin E, vitamin K, thiamin, riboflavin, niacin, vitamin B6, folate, vitamin B12, biotin, pantothenic acid, and phosphorous.

11. A method for making a coated mineral-amino acid/polysaccharide complex, wherein the method comprises:
dry-blending a combination of (a) one or more mineral-amino acid compounds having a particle size smaller than about 80 mesh with (b) a polysaccharide and (c) a slow release coating material;
granulating the dry-blended combination to generate a granulated material; and
drying the granulated material.

12. The method of claim 11, wherein the drying comprises using a fluid bed dryer.

13. The method of claim 11, further comprising wet milling the granulated material to reduce the particle size of the material to 10 mesh or smaller.

14. A composition comprising a first compound that comprises a first mineral coupled to a first amino acid, wherein the first compound is coated with a slow release coating.

15. The composition of claim 14, wherein the first mineral is selected from the group consisting of zinc, copper, selenium, manganese, chromium, and iron.

16. The composition of claim 14, wherein the first amino acid is selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine.

17. The composition of claim 14, wherein the slow release coating comprises sodium alginate, stearic acid, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, monoglycerides, diglycerides, or any combination thereof.

18. The composition of claim 14,
wherein the first compound is selected from the group consisting of:
a zinc-amino acid,
a copper-amino acid,
a selenium-amino acid,
a manganese-amino acid,
a chromium-amino acid, and
an iron-amino acid.

19. The composition of claim 14, further comprising one or more of vitamin A, vitamin C, vitamin D, vitamin E, vitamin K, thiamin, riboflavin, niacin, vitamin B6, folate, vitamin B12, biotin, pantothenic acid, and phosphorous.

20. The composition of claim 14, further comprising a second compound that comprises a second mineral coupled to a second amino acid, wherein the second mineral is different from the first mineral, and wherein the second compound is not coated with a slow release coating.
